(19) United States
(12) Patent Application Publication
(10) Pub. No.: US 2010/0292706 A1
(43) Pub. Date: Nov. 18, 2010

(54) NOVEL ENHANCED HAPTIC FEEDBACK PROCESSES AND PRODUCTS FOR ROBOTIC SURGICAL PROSTHETICS

(75) Inventors: Erik P. Dutson, Los Angeles, CA (US); Warren Grundfest, Los Angeles, CA (US); Adrienne Higa, Los Angeles, CA (US); Chih-Hung King, Sharpsburg, GA (US); Martin Culjat, Los Angeles, CA (US)

Correspondence Address: JOHN P. O'BANION;O'BANION & RITCHEY LLP, 400 CAPITOL MALL SUITE 1550, SACRAMENTO, CA, 95814, US

(73) Assignee: THE REGENTS OF THE UNIVERSITY CALIFORNIA

(21) Appl. No.: 12/279,982

(22) PCT Filed: Apr. 14, 2007
(86) PCT No.: PCT/US2007/066677

§ 371 Date: Apr. 22, 2010

Related U.S. Application Data
(60) Provisional application No. 60/791,960, filed on Apr. 14, 2006.

(51) Int. Cl. A61B 19/00

(52) U.S. Cl. 606/130

(57) ABSTRACT

A modular, scalable, layerable balloon actuator or actuator array. The miniaturized actuator array can be mounted on the hand controls of a surgical robotic system, and pressure or force input is applied to the surgeon's fingers. The input to the fingers is proportional to the applied force or pressure that is sensed on a separate sensor array, which is mounted on the surfaces of the object to be physically manipulated. The force is translated to pressure using a control system, which includes electronic and pneumatic components. The Novel enhanced haptic feedback system enables the detection of force and tactile information on tissues and sutures with high spatial and temporal resolution. This technology shortens the learning curve for MIS training, expands the application of MIS techniques in surgery, and enhances telementoring and teiesurgery applications. The actuator is modular scalable, Iayerable, compact, configurable, flexible, and conformable. It is therefore designed such that it can be adapted to future surgical robotic systems, and can be applied to prosthetics, orthotics, and persons with sensory neuropathy, as well as other robotic applications, simulating machines and apparatus and user-interfacing systems for video-gaming.

### GOVERNMENT SPONSORSHIP

Aspects of the instant disclosure were sponsored by that certain Telemedicine and Technology Research Center for the Army of the United States of America (TATRC) Contract, denoted W81XWH-05-2-0024, wherein the Principal Investigator is the first named inventor of the instant patent application. Likewise, the Center for Advanced Surgical and Interventional Technologies (CASIT) was initiated both by philanthropic donative instruments and TATRC funding.

### BACKGROUND OF THE DISCLOSURE

The present disclosure relates to improved tactile feedback systems, products, process and methods and medical devices for enhancement of tactile feedback. In particular, the present disclosure relates to optimized haptic interfacing processes and products thereby.

Minimally invasive surgery (MIS) has revolutionized surgical care and treatment, reducing trauma to the patient, decreasing the need for pain medications, and shortening recovery times and hospital stays. MIS has been used in the military and is also proposed for battlefield surgery pods. One drawback of current laparoscopic techniques is the reduction of tactile, or haptic, feedback to the surgeon. This has likely limited the expansion of MIS applications and contributed to an increased learning curve for surgeons. Robotic MIS offers improved range of motion and other technical advantages, but is characterized by a total loss of haptic feedback.

Those skilled in the art readily understand that numerous desiderata relating to the “hand” or feel of remotely actuated devices have created a series of longstanding needs. The instant disclosure offers for consideration numerous aspects which artisans shall embrace as new ways to address these traditional surgical issues and other things. According to the instant teachings, improved tactile feedback enables numerous automatic and robotic systems to function at a new level by enriching the user interface for prosthetics, orthotics, video-games and simulations useful from applications ranging from sensory neuropathy addressing devices to new filmed audio and video driven systems.

### SUMMARY OF THE DISCLOSURE

Briefly stated, the invention includes a modular, scalable, layerable balloon actuator or actuator array. The miniaturized actuator array is, in one embodiment, mounted on the hand controls of a surgical robotic system, and pressure or force input is applied to the surgeon's fingers. The input to the fingers is proportional to the applied force or pressure that is sensed on a separate sensor array, which is mounted on the surfaces of the object to be physically manipulated.

According to embodiments of the present invention, there is provided an improved pneumatic tactile system comprising in combination, at least an actuator, sensors operatively linked to the at least an actuator and a control system for regulating input in proportion to applied force and pressure, whereby a latency period between movement of the user and feedback transmitted to equipment spans a time period of less than at least about 300 milliseconds.

According to the further embodiments of the present invention there is provided a pneumatic tactile apparatus comprising, in combination, a substrate, balloon membrane mounted between a user interface and an apparatus to be manipulated.

According to still further embodiments of the present invention there is provided a haptic feedback system which is wireless and scalable comprised of a sensor array, a system controller and a plurality of pneumatically controlled actuators, wherein an air source is a gas.

The force is translated to pressure using a control system; which includes electronic and pneumatic components. These components (actuator, sensor, and control system) comprise a Haptic Feedback System. This haptic feedback system can enable, the detection of force and tactile information on tissues and sutures with high spatial and temporal resolution.

This technology shortens the learning curve for MIS training, expands the application of MIS techniques in surgery, and enhances telementoring and telesurgery applications. The actuator is modular scalable, layerable, compact, configurable, flexible, and conformable. It is therefore readily adaptable to future surgical robotic systems, and can be applied to prosthetics, orthotics, and persons with sensory neuropathy, as well as other robotic applications, simulating machines and apparatus and user-interfacing systems for video-gaming.

According to a feature of the present disclosure there is provided an improved pneumatic tactile system, which comprises in combination a substrate and a balloon membrane to be mounted between a user interface and an apparatus to be manipulated, wherein the apparatus to be manipulated is at least one of an orthotic and a prosthetic.

According to another feature of the instant haptic feedback system which is wireless and scalable comprising a sensor array, a system controller, a plurality or array of pneumatically controlled actuators, and at least a wireless controller, whereby the process is used with at least one of a video-gaming system and a simulation based on audio and video input and feedback.

### DRAWINGS

The above-mentioned features and objects of the present disclosure will become more apparent with reference to the following description taken in conjunction with the accompanying drawings wherein like reference numerals denote like elements, to the extent feasible and consistent wherein:

FIG. 1 is a generalized schematic showing a system according to teachings of the present invention;

FIG. 2 is an engineering schematic of embodiments according to the teachings of the present invention;

FIG. 3 is another detailed engineering schematic showing aspects of the instant system; and

FIG. 4 is a view of one version of a user interface according to the present invention.

### DETAILED DESCRIPTION

It will be understood that various details of the presently disclosed subject matter may be changed without departing from the scope of the presently disclosed subject matter. Furthermore, the foregoing description is for the purpose of illustration only, and not for the purpose of limitation. Artisans will readily grasp use of the instant teachings in their respective fields, from minimally invasive surgery to robotics to filmed entertainment and video-gaming.

The present inventors have discovered that lack of haptic, or force, feedback limits the surgeon's ability to apply robotically assisted surgical systems and complex laparoscopic techniques to technically demanding procedures. Without haptic feedback, surgeons must rely primarily on visual cues, and are thus deprived of their tactile senses when tying sutures and manipulating tissues. This has delayed advancement of the field, and underscores the need for improved user interface systems which “feel” like the process they are replacing.

Likewise, those involved in simulations, video gaming, filmed entertainment and related industries have needs addressed by the instant system. Artisans will understand readily how to use the instant system for applications in their respective field of use. For example, virtual reality-based gaming systems allowing users to “sense” based on their actions have needed better haptic feedback for some time.

During robotic surgery, surgeons must rely on visual cues alone. This likely contributes to longer learning curves for MIS procedures, and decreases the surgeon's ability to detect tissue characteristics, which can lead to inadvertent tissue damage and surgical errors. Addition of haptic feedback capabilities to complex laparoscopic and robot-assisted surgical systems improves the quality and safety of surgical procedures and allows for expansion of these techniques to other applications.

To address the feedback limitations of current robot-assisted surgical systems, the development of an adaptable, reliable, scalable, and affordable haptic feedback system is offered for consideration by the instant systems. The concept of haptic feedback and its application to MIS have previously been investigated; however, current technologies have limited applicability to existing laparoscopic and robotic tool systems. These limitations include excessive manufacturing costs, bulky and complicated designs, and long learning curves. Previously developed haptic feedback devices for robotic surgical systems require redesigning and reengineering of the systems themselves, greatly increasing cost and complexity of the final integrated system designs.

A pneumatic balloon-based haptic feedback system has previously been proposed by another group for laparoscopic surgery; however the actuator design is impractical for attachment onto laparoscopic or robotic tools due to its bulky design and lack of modularity, scalability, uniformity, and layerability as has been detailed in the literature. Artisans can access hundreds of articles explaining the shortcomings of known systems, thus, further detail regarding the same is omitted from this discussion. However, exemplary references are listed for this purpose.

Key benefits of the instant system, according to embodiments of the present invention include scalability and adaptability to various laparoscopic and robotic surgical tools, as well being easily and practically combined with other robotic or prosthetic applications. By developing a scalable and modular wireless haptic feedback system, complicated system redesigns and high system integration costs can be avoided and teaming curves are shortened.

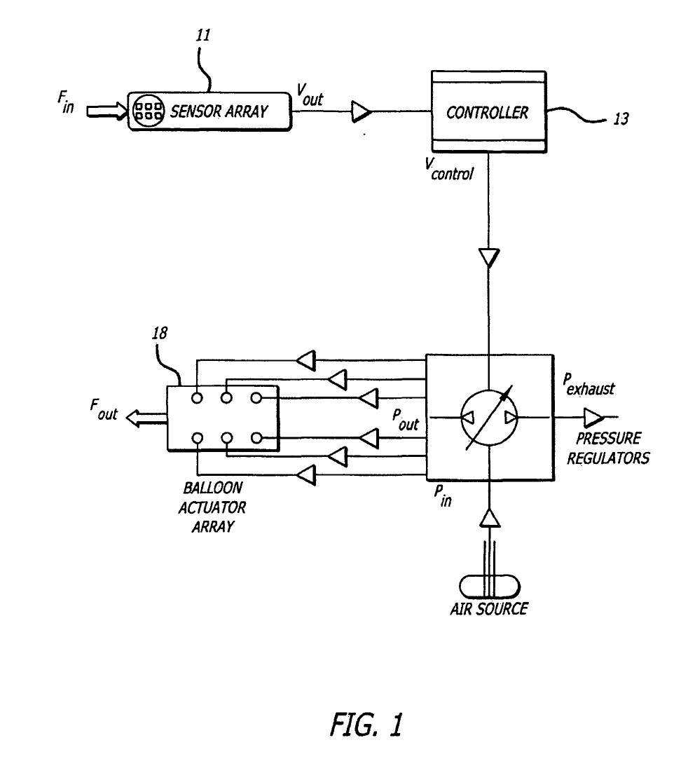

Referring to FIG. 1 through FIG. 4, a FlexiForce (Tekscan, Inc.) A201 piezoresistive sensor 11 has been selected as an exemplary force sensor, according to an illustrative, but not limiting, embodiment. Upon application of a force or pressure to the sensor surface, a proportional voltage change is detectable. A microcontroller unit 13 has been programmed to translate the voltage input from sensor 11 to a proportional pressure output, which will actuate a pneumatic balloon. Various prototype balloon actuators have been manufactured from, for example, Soda Clear Dragon Skin brand of silicone rubber film (Smooth-on®, Inc.) and macromolded polydimethylsiloxane (PDMS) base.

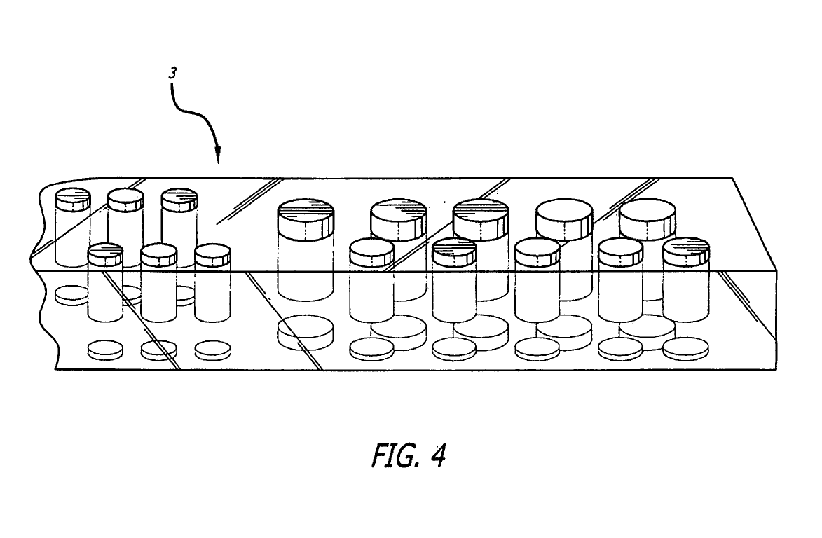

Testing has demonstrated a maximum actuation pressure of 15 psi over 75 actuation cycles for a 300 μm thick membrane. Investigators in research groups were able to consistently distinguish between three actuation levels over the 15 psi range. Artisans readily understand these parameters, and how they relate to the instant systems; as shown in FIG. 4, user interface 3 allows the operator to react and to act on forces transmitted through various depicted system embodiments.

Membranes have been fabricated with thicknesses ranging from 100 μm to 500 μm, either in a single or multi-layer configuration. Substrates have been fabricated with various arrays, channels, and tubing configurations and dimensions—all a result of the modularity and scalability of the actuator design. Surgeons have already mastered use of the instant system and use it for numbers of procedures, in addition to training and education applications developed.

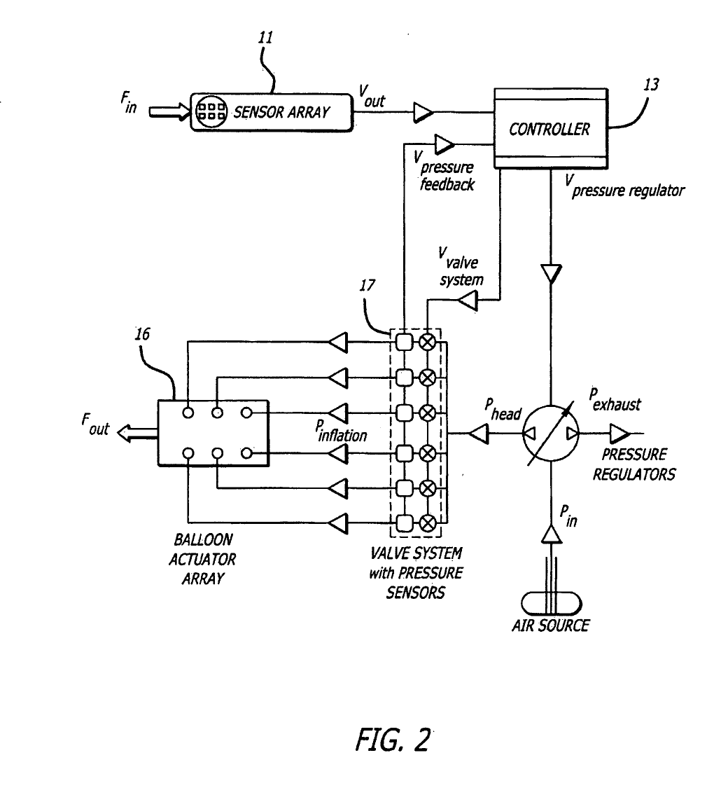

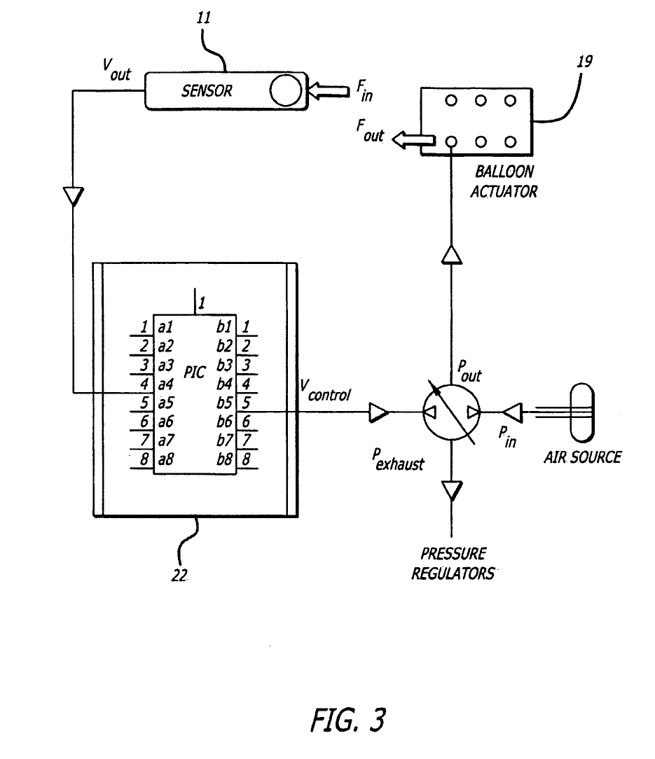

FIG. 2 and FIG. 3 shows an alternate design which is a more complex prototype consisting of multi-element sensor 11 and actuator arrays 16, 17. This added complexity improves the resolution of the system and will also allow the force sensor to act as a slip sensor, measuring shear as well as compressive forces; that is, the detection of objects or tissues slipping from the grasper, in one embodiment. The end result being that the instant system has a “hand” or “feel” allowing the user to operate the system as if there were nothing between the user and the object to be manipulated. Referring now to FIG. 3, also the skilled in the art will understand based upon the foregoing discussion, figures and the claims which are appended hereto, how input from sensor becomes output from sensor traveling through chip 22, which may be any customized or designed element as is available to then transfers output as depicted.

According to embodiments, for example, like that shown in FIG. 2, micro fabrication of the instant teachings using micro-electro-mechanical systems (MEMS) technology has been accomplished. Psychomotor testing validated and enables the team to optimize the balloon array characteristics, including the balloon diameter, spacing, inflation pressure and maximum deflection. In their way, surgery, telementoring has become possible with use of interface 3.

According to embodiments of these inventions, an optimized haptic feedback system is effective to be retrofitted onto the robotic surgical instruments for in vitro and in vivo clinical testing, on an ongoing basis. It is respectfully proposed that the instant improvements over the state of the art constitute progress in science and the useful arts, and permit users to have haptic input making many tasks easier.

While the apparatus and method have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

### REFERENCES

The references listed below are incorporated herein by reference only as appropriate under National law, per the Paris Convention to the extent that they supplement, explain, provide a background for or teach methodology, techniques and/or processes employed herein. All cited publications referred to in this application are herein expressly incorporated by reference, as appropriate under national law mindful that those skilled in the art are aware of those systems described and how the present invention relates to and interfaces with them.

Feussner, H., Siewert, J R., “Reduction of surgical access trauma: reliable advantages.” Chirug 2001 March 72(3):236-44.

Jacobs, J K, Goldstein, R E, “Laparoscopic adrenalectomy: a new standard of care” Annals of Surgery 1997 May; 225(5) pp 495-501.

Rosen J. Hannford B, “Doc at a distance,” IEEE Spectrum, October 2006, 34-39.

Salle, D., Gosselin, F., Bidaud, P., Gravez, P., “Analysis of haptic feedback performances in telesurgery robotic systems”, IEEE International workshop on Robot and Human Interactive Communication, 2001.

Jacob, B., Gagner, M., “Robotics and General Surgery”, Surgical Clinics of North America, Vol 83 Number 6 December 2003, pp 1405-1419.

Hayward, V., Cruz-Hernandez, M, “Tactile Display Device Using Distributed Lateral Skin Stretch,” Proc. Haptic Interfaces for Virtual Environment and Teleoperator Systems Symposium, ASME IMECE2000, Orlando, Fl., USA. Proc. ASME DSC-69-2, pp. 1309-13 14.

Kontarinis D A, Son J S, Peine W, Howe R D, “A tactile shape sensing and display system for teleoperated. manipulation,” IEEE Int. Conf. on Robotics and Automation, Nagoya, Japan, 2 1-27 May 1995. IEEE, Robot and Automat Soc, 1995, pp. 641-646.

Hannaford B, Trujillo J, Sinanan M, Moreyra M, Rosen J, Brown J, Lueschke R, MacFarlane M, “Computerized endoscopic surgical grasper,” in MMVR-98, Jan. 1998, pp. 111-117.

Okamura A M, Webster R J, Nolin J T, Johnson K W, and Jafry H, “The Haptic Scissors: Cutting in Virtual Environments,” in Proc. IEEE Int. Conf. Rob. Autom., 2003, pp. 828-83 3.

Taylor R, Jensen P, Whitcomb L et al, “A steadyhand robotic system for microsurgical augmentation,” Int. J. Robotic. Res., 1999;18:12.

Caldwell, D G Tsagarakis N, Giesler, C, “An Integrated Tactile/Shear Feedback Array for Stimulation of Finger Mechanoreceptor”, IEEE Int. Conf. Rob. and Auto., vol 1 pp. 287-292, Detroit, Mich., May 1999.

Brown J D, Rosen J, Chang L, Sinanan M N, Hannaford B, “Quantifying surgeon grasping mechanics in laparoscopy using the blue DRAGON system,” Studies in Health Technology and Informatics—Medicine Meets Virtual Reality, Newport Beach, Calif.,

Sherrick C E, Cholewiak, “Cutaneous sensitivity,” Handbook Percep Human Perf v.1: Sensory Processors and Perception, John Wiley & Sons, 1986.

Schiff W, Foulke E, Tactual Perception: A Sourcebook, Cambridge: Cambridge University Press, 1982, chap 2.

Tiresias.org, “Braille cell dimenstions,” http://www.tiresias.org/reports/braille cell.htm

Toshio Fukuda, Hideyuki Morita, Fumihito Arai, Hidenori Ishihara, Hideo Matsuura, “Micro resonator using electromagnetic actuator for tactile display”, International Symposium on micromechatronics and human science; 143-148, (1997)

Darwin G. et at “An integrated tactile/shear feedback array for stimulation of finger mechanoreceptor”, Proc 1999 IEEE Intl Conf Rob Autom, Detroit, Mich., 187-192 (1999).

Brown J D, Rosen J, Kim Y S, Chang L, Sinanan M N, Hannaford B, “In-vivo and in-situ compressive properties of porcine abdominal soft tissues,” Studies in Health Technology and Informatics—Medicine Meets Virtual Reality, Newport Beach, Calif., January 2003.

De Visser H, Heijnsdijk E A M, Herder J L, Pstecky P V, “Forces and displacements in colon surgery,” Surg Endos, 16: 1426-30, (2002).

Okamura A, Simone C, O′Leary M, “Force modeling for needle insertion into soft tissue,” IEEE Trans Biomed Eng, 51 (10): 1707-15, (2004).

Brouwer I et al, “Measuring in vivo animal soft tissue properties for haptic modeling in surgical simulation,” Medicine Meets Virtual Reality, 2001.

Jungmann, M, Schlaak, H. “Miniaturized Electrostatic Tactile Display with High Structural Compliance.” Eurohaptics 2002, Edinburgh, U.K., 2002

Goodwin A W, Macefield V G, Bisley J W, “Encoding of object curvature by tactile afferents from human fingers,” J Neurophys, 78 (6): 288 1-2888, 1997.

King C H, Higa A T, Culjat M O, Han S H, Bisley J W, Carman G P, Dutson E, Grundfest W S, “A pneumatic haptic feedback actuator array for robotic surgery or simulation,” Proceedings of Medicine Meets Virtual Reality 15: in vivo, in vitro, in silico: Designing the next in Medicine, 6-9 Feb. 2007, Long Beach, Calif., 2 17-222, 2007.

Prasad S., Kitagawa M., Fischer G., Zand J., Talamini M., Taylor R., Okamura A., “A Modular 2-DOF Force-Sensing Instrument For Laparoscopic Surgery.” Sixth International Conference on Medical Image Computing and Computer Assisted Intervention (MICCAI), 2003.

Dario P., Hannaford B., Menciassi A., “Smart Surgical Tools and Augmenting Devices.” IEEE Trans. on Robotics and Automation, 2003;19(5):782-792.

Tholey, G., Desai, J., Castellanos, A., “Force feedback plays a significant role in minimally invasive surgery: results and analysis.” Annals of Surgery Volume. 241(1), January 2005, pp 102-109.

Rosen, J., Hannaford, B., MacFarlane, MP., Sinanan, MN., “Force Controlled and Teleoperated Endoscopic Grasper for Minimally Invasive Surgery-Experimental Performance Evaluation.” IEEE Trans. Biomed. Eng. 1999 October;46(10):1212-21.

Chanter C, Summers I., “Results from a Tactile Array on the. Fingertip” Proceedings of Eurohaptics 2001, pp. 26-28, Birmingham, UK, 2001.

Satava, R., Bowersox, J., Mack, M., Krummel, T., (2001) “Robotic surgery: state of the art and future trends.” Contemp Surg 57:489-500.

Moy, G., Wagner, C., Fearing, R. S., “A compliant tactile display for teletaction,” presented at Proceedings 2000 ICRA. IEEE Intl. Conf. on Robotics and Automation, Piscataway, N.J., USA, 2000.

Marescaux, J., Leroy, J., Gagner, M., Rubino, F., Mutter, D., Vix, M,. Butner, S E., Smith, M K., “Transatlantic robot-assisted telesurgery” Nature 2001 Sept 27;413(6854):379-80.

Smooth-On, Inc., http://www.smooth-on.com.

Pawluk, D., van Buskirk, C., Killebrew, J., Hsiao, S., Johnson, K., “Control and Pattern Specification for a High Density Tactile Array”, IMECE Proc of the ASME Dyn Sys and Control Day , vol 64, pp 97-102, Anaheim, Calif., November 1998.

Wagner, C., Lederman, S., Howe, R., “Design and Performance of a Tactile Shape Display Using RC Servomotors.” Journal of Haptics Research 2004.

Jacobs-Cook A. J., “MEMS versus MOMS from a systems point of view,” J. Micromech. Mricoeng., vol 6, pp 148-156, 1995.

Tekscan, Inc., http://www.tekscan.com/flexiforce/specs flexiforce.html.

1. An improved pneumatic tactile system, which comprises in combination:
at least an actuator;
sensors operatively linked to the at least an actuator; and
a control system for regulating input in proportion to applied force and pressure;
whereby a latency period between movement of a user and feedback transmitted to equipment spans a time period of less than at least about 300 milliseconds.

2. A pneumatic tactile apparatus, which comprises in combination; a substrate; and a balloon membrane to be mounted between a user interface and an apparatus to be manipulated.

3. The pneumatic tactile apparatus of claim 2, the apparatus further comprising:
a substrate which can easily adjust material properties independent from membrane.

4. The pneumatic tactile apparatus of claim 2, the apparatus further comprising:
a membrane which can easily adjust material properties independent from the substrate.

5. The pneumatic tactile apparatus of claim 2, further comprising: a variable set of configurations, element spacings, diameters, architectures, and dimensions.

6. The pneumatic tactile apparatus of claim 2, the material of which can be rigid, deformable, elastic, plastically deformable and combinations of the these conditional states.

7. The pneumatic tactile apparatus of claim 2, whereby at least one of the device, substrate and the membrane are layered.

8. The pneumatic tactile apparatus of claim 2, wherein the membrane may be a layer of the substrate.

9. The pneumatic tactile apparatus of claim 2, the layers of which can be made in at least one of a single process, multiple processes and by process whereby various sections can be added together; and wherein interconnects/IC/MEMS/sensors/devices are integrated into the substrate and membrane and combinations of the same.

10. A haptic feedback system which is wireless and scalable comprising:
a sensor array;
a system controller; and
a plurality of pneumatically controlled actuators; wherein an air source for the pneumatic tactile system can be any gas selected from the group consisting of CO₂, compressed/pumped air, Nitrogen, Helium, Oxygen and combinations thereof.

11. The haptic feedback system of claim 10, said actuator further comprising a source of actuation being at least one of liquid-based and plasma-based.

12. The haptic feedback system of claim 10, further comprising the actuator being a device inflating in response to stimuli selected from the group consisting of video and audio input.

13. The haptic feedback system of claim 10, wherein the actuator further comprises a device inflating in response to preprogrammed stimuli further comprising at least one of a preprogrammed algorithm set and data and stimuli-based information generated by the system, and combinations of the same.
